(19) United States
(12) Patent Application Publication
(10) Pub. No.: US 2022/0401138 A1
(43) Pub. Date: Dec. 22, 2022

(54) EXPANDABLE BONE FIXATION SCREW WITH REMOVABLE CORE PIN

(71) Applicant: Nextremity Solutions, Inc., Warsaw, IN (US)

(72) Inventors: Adam FINLEY, Warsaw, IN (US); Thomas HOLTON, Winona Lake, IN (US); Joel CARNE, Warsaw, NY (US)

(73) Assignee: Nextremity Solutions, Inc., Warsaw, IN (US)

(21) Appl. No.: 17/659,927

(22) Filed: Apr. 20, 2022

Related U.S. Application Data
(60) Provisional application No. 63/213,269, filed on Jun. 22, 2021.

(51) Int. Cl. A61B 17/86

(52) U.S. Cl. CPC A61B 17/8605

(57) ABSTRACT

A bone screw, and method of using the same, are described. The bone screw includes a head, a distal threaded portion, and an expandable intermediate portion. The intermediate portion is expandable in response to a force being applied from an unstressed or relaxed state to a stressed or unrelaxed state. A removable core pin may be inserted into the intermediate portion to stretch or stress and/or stabilize the intermediate portion.

### CROSS REFERENCE TO RELATED APPLICATIONS

The present application claims priority to U.S. Provisional Application Ser. No. 63/213,269 filed on Jun. 22, 2021 (Attorney Docket No. 3768.112P1), which is incorporated herein by reference in their entireties

### BACKGROUND OF THE INVENTION

### Technical Field

The present invention generally relates to bone screws and, more particularly, to a bone fixation screw including an expandable portion and a removable core pin.

### Background Information

Orthopedic fastening devices, such as sutures, pins, screws and the like, are typically used in the treatment of many bone fractures. Such fasteners are used to hold distal and proximal bone fragments in accurate alignment with each other with the hope that the fragments will be sufficiently close together to permit the rapid bonding or fusing of the fragments together. These fasteners may also be used with orthopedic implants and orthopedic appliances such as, for example, a bone plate.

Conventional fasteners, however, have significant disadvantages when used in certain situations requiring compression of bones or fractures, such as, for example, ankle syndesmosis, LisFranc, joint fusions, and light fracture fixation. The specific amount of compression applied in these types of applications is critical. For example, when treating ankle syndesmosis, not enough compression leads to instability and over compression leads to subluxation of, for example, the talus.

The use of sutures is undesirable in some situations because sutures are too flexible and will not result in healing of soft tissue. Conventional screws and pins are too rigid, potentially causing fatigue and breakage. When a bone screw is employed, either to fasten two or more bone fragments together or to secure an orthopedic appliance (e.g., bone plate) to a bone surface, and the bone screw is tightened, initially, tension in the screw is relatively very high, and holds the bone fragments together. However, bone is a viscoelastic material and undergoes a phenomenon known as stress relaxation immediately after torque has been applied to the bone screw. The stress relaxation response is quite pronounced and causes immediate and rapid reduction in the bone screw tension and, hence, the force holding the bone fragments together. Furthermore, after a conventional bone screw is tightened, and the bone fragment is laterally displaced, as by bending, the rigidity of the bone screw causes the surrounding bone to fail because the bone has lower strength and stiffness than the bone screw. This can lead to failure of the fixation and eventual non-union or misalignment of the bone fragments at the fracture site.

One approach to overcome this problem has been the use of a bone screw having a helical portion, as described in U.S. Pat. No. 6,656,184 to White et al. In this reference, a conventional bone screw is modified to include an intermediate helical portion that requires the use of a resorbable material within the spaces of the helical portion when the helical portion is in a stressed state. The resorbable material is used to slowly induce transition of the pretention load after insertion into the desired location, e.g. fracture or joint. However, the use of a resorbable material has significant disadvantages, including, for example, the brittleness of the resorbable material and the unknown forces being applied to the bone at various points in time. Also, a bone screw having a helical portion relying on a resorbable material is not strong enough to initially insert into the desired bone location.

Thus, a need exists for a bone fixation screw that provides, for example, more stability than a suture construct and more flexibility than a conventional screw. There is also a need for a bone fixation screw that further provides, for example, a quantifying and known amount of compression force across the fracture at predetermined and predictable levels.

### SUMMARY OF THE INVENTION

Briefly, a bone fixation screw constructed in accordance with one or more aspects of the present invention provides, for example, controlled and repeatable compression, the ability to work on its own or with plates and/or nails, stable fixation with limited flexibility and a simple and quick implant fixation system.

In one embodiment, a bone fixation screw constructed in accordance with one or more aspects of the present invention includes, for example, an expandable portion that could be stretched to a known force value and held with a removable core pin in the cannulation of the screw. The bone fixation screw would be provided to a surgeon in this form. The bone fixation screw could then be inserted across, for example, the fracture or joint. Once in place, the core pin is removed and the pre-tension load is transitioned across the fracture or joint at the predetermined level. Quantifying the amount of compression that is being induced across the fracture or joint is becoming more desirable so that a user does not under or over compress the fracture or joint, especially with ankle syndesmosis. A bone fixation screw constructed in accordance with one or more aspects of the present invention allows a user to apply a known compression force every time.

In one embodiment, a shorter core pin may be inserted to change the stiffness of the bone fixation screw after being implanted.

These, and other objects, features and advantages of this invention will become apparent from the following detailed description of the various aspects of the invention taken in conjunction with the accompanying drawings.

### BRIEF DESCRIPTION OF THE DRAWINGS

The present invention will be understood more fully from the detailed description given hereinafter and from the accompanying drawings of the certain embodiments of the present invention, which, however, should not be taken to limit the invention, but are for explanation, illustration and understanding only.

FIG. 1 depicts a perspective view of a bone fixation screw constructed in accordance with one or more aspects of the present invention;

FIG. 2 depicts a perspective view of one embodiment of a core pin constructed in accordance with one or more aspects of the present invention;

FIG. 3 depicts an exploded view of the bone fixation screw depicted in FIG. 1;

FIG. 4 depicts a side cross-sectional view of a bone fixation screw in a relaxed or unstressed state without a core pin constructed in accordance with one or more aspects of the present invention;

FIG. 5 depicts a side cross-sectional view of a bone fixation screw in a stressed or unrelaxed state without a core pin constructed in accordance with one or more aspects of the present invention;

FIG. 6 depicts a side cross-sectional view of a bone fixation screw in a stressed or unrelaxed state with a core pin constructed in accordance with one or more aspects of the present invention;

FIG. 7 depicts a side cross-sectional view of a bone fixation screw in a stressed or unrelaxed state without a shorter core pin constructed in accordance with one or more aspects of the present invention;

FIG. 8 depicts a partial side view of a bone fixation screw illustrating one embodiment of an expandable portion of an intermediate portion including multiple ribbons extending between a head portion and a distal portion constructed in accordance with one or more aspects of the present invention;

FIG. 9 depicts a perspective view of another embodiment of a core pin constructed in accordance with one or more aspects of the present invention;

FIG. 10 depicts a side cross-sectional view of anther embodiment of a bone fixation screw without a core pin inserted constructed in accordance with one or more aspects of the present invention;

FIG. 11 depicts an application of a bone fixation screw constructed in accordance with one or more aspects of the present invention used to correct an ankle syndesmosis injury;

FIG. 12 depicts an application of a bone fixation screw constructed in accordance with one or more aspects of the present invention used to correct a distal tibia fracture; and

FIG. 13 depicts a cross-sectional side view of an alternative embodiment of a bone fixation screw constructed in accordance with one or more aspects of the present invention.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth to provide a thorough understanding of the present invention. It will be obvious, however, to those skilled in the art that the present invention may be practiced without some of these specific details. In other instances, well-known structures are not shown in detail to avoid unnecessary obscuring of the present invention.

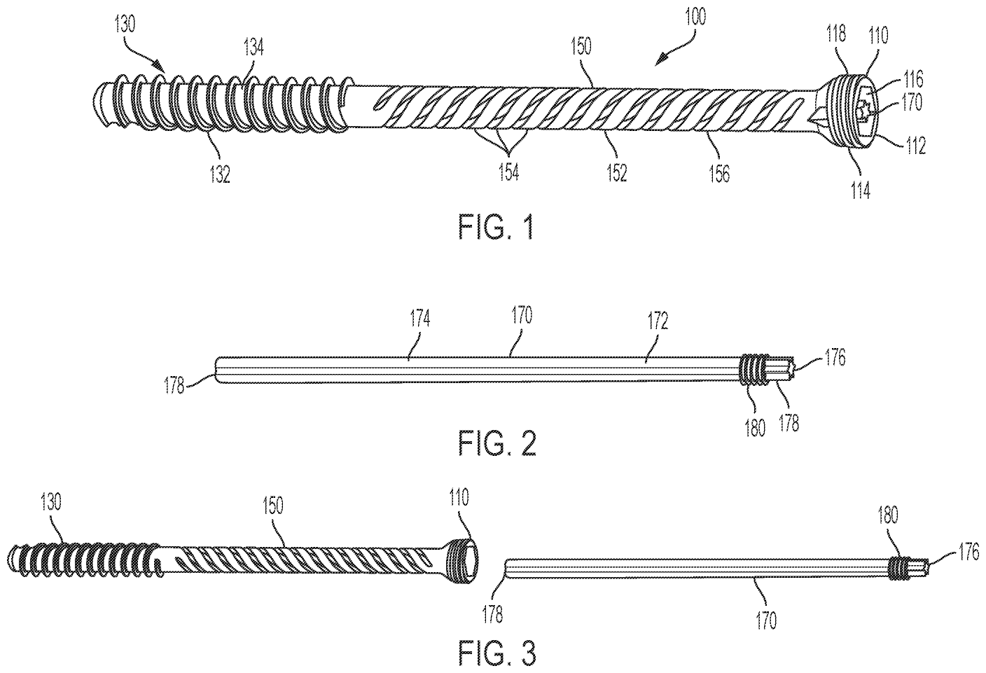

Thus, all implementations described below are exemplary implementations provided to enable persons skilled in the art to make or use the embodiments of the disclosure and are not intended to limit the scope of the disclosure, which is defined by the claims. As used herein, the word “exemplary” or “illustrative” or “example”, and derivatives thereof, means “serving as an example, instance, or illustration.” Any implementation described herein as “exemplary” or “illustrative” or “example”, and derivatives thereof, is not necessarily and should not be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding technical field, background, brief summary or the following detailed description. It is also understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise. While this invention is satisfied by embodiments in many different forms, there is shown in the drawings, and will herein be described in detail, one or more embodiments of the present invention with the understanding that the present disclosure is to be considered as exemplary of the principles and aspects of the invention and is not intended to limit the invention to the embodiments illustrated. The scope of the invention will be pointed out in the appended claims.

In short, a bone fixation screw constructed in accordance with one or more aspects of the present invention provides, for example, controlled and repeatable compression, the ability to work on its own or with plates and/or nails, stable fixation with limited flexibility and a simple and quick implant fixation system. A bone fixation screw constructed in accordance with one or more aspects of the present invention includes an expandable portion that could be stretched to a known force value and held with a removable core pin in the cannulation of the screw. The bone fixation screw would be provided to a surgeon in this form. The bone fixation screw would then be inserted across, for example, the fracture or joint. Once in place, the core pin is removed and the pre-tension load is transitioned across the fracture or joint at the predetermined level. Quantifying the amount of compression that is being induced across the fracture or joint is becoming more desirable so that a user does not under or over compress the fracture or joint, especially with ankle syndesmosis. A bone fixation screw constructed in accordance with one or more aspects of the present invention allows a user to apply a known compression force every time. In one embodiment, a shorter core pin may be inserted to change the stiffness of the bone fixation screw.

FIGS. 1-3 illustrate one embodiment of a bone fixation screw 100 constructed in accordance with one or more aspects of the present invention. As illustrated, bone fixation screw includes a head portion 110, a distal portion 130, an intermediate portion 150 positioned between head portion 110 and distal portion 130, and one or more core pin(s) 170.

Distal portion 130 may include a threaded surface 132 formed on its outer surface 134. Threaded surface 132 may have a right-hand or left-hand turning orientation.

Head portion 110 includes an end surface 112 and a side surface 114. End surface 112 includes an opening to a recess 116 formed in head portion 110. In one example, recess 116 may be hex shaped for receiving a driving device, such as, for example, a hex driver. Although recess 116 is shown as being hex-shaped, it is envisioned that other configurations may be employed as well, such as different recess shapes or differently shaped male members to be engaged by a female drive mechanism. Head portion 110 may be a locking head that includes, for example, threads 118 on side surface 114 to, for example, attach to a bone plate.

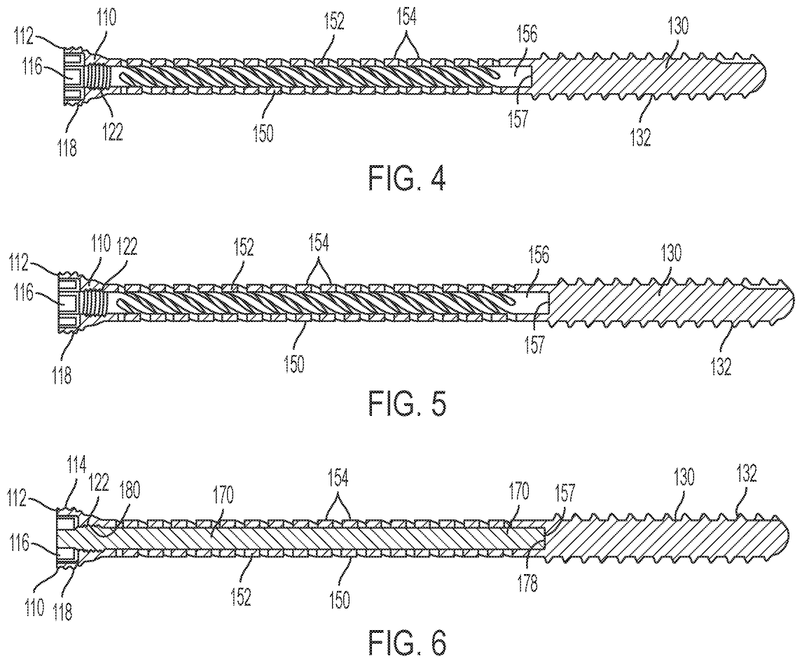

Intermediate portion 150 includes a compressive member such as, but not limited to, an expandable portion 152. In one embodiment, expandable portion 152 may include a single helix 154, formed therein by any number of conventional methods, such as, but not limited to, wire electrical discharge machining. In an alternative embodiment, illustrated in FIG. 8, expandable portion may include, for example, a double or more helix design having multiple ribbons extending between head portion 110 and distal portion 130. In one embodiment illustrated in FIG. 8, the helix design may use multiple starts extending from head portion 110 that include multiple ribbons 154a-154d extending between head portion 110 and distal portion 130. In one such embodiment, ribbons 154a-154d may be loaded equally so if one of the ribbons would fail, then the other ribbons would remain intact and allow bone fixation screw 100 to still be removed, if necessary. Intermediate portion 150 also defines an inner bore 156 (see e.g. FIG. 4) extending from, and in communication with, recess 116 formed in head portion 110 to distal portion 130.

Core pin 170 is removably inserted into inner bore 156 of intermediate portion 150 through recess 116 in proximal head portion 110. Core pin 170 provides stability to bone fixation screw 100 as it is inserted into bone. As illustrated in FIG. 2, in one example, core pin 170 includes a tubular body 172 sized to fit within inner bore 156 of intermediate portion 150. Tubular body 172 includes an outer surface 174, a first end 176 and a second end 178. First end 176 of tubular body 172 includes a head 178 shaped to assist in inserting core pin 170 into inner bore 156 of intermediate portion 150. In one example, as illustrated, head 178 may be star shaped, although other shapes may be employed. At least a portion of outer surface 174 near first end 176 may include a first set of threads 180. First set of threads 180 may mate with threads 122 formed in the inner surface of inner bore 156 prior to the start of expandable portion 152 to, for example, retain core pin 170 within inner bore 156.

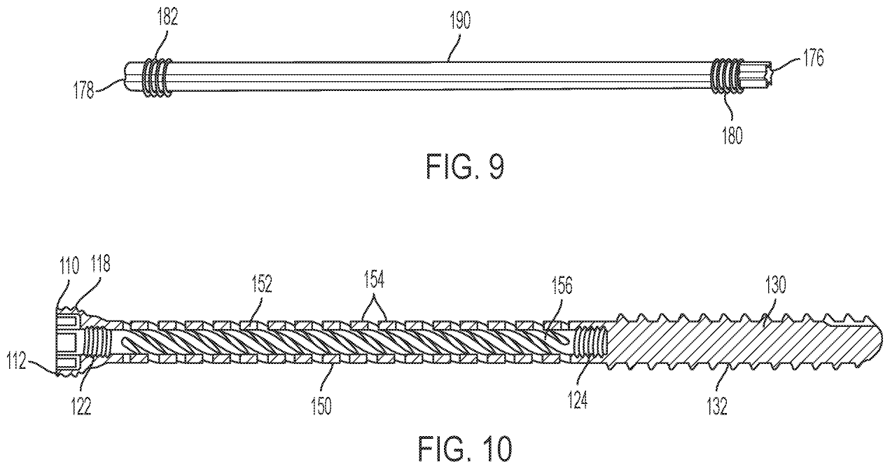

In an alternative embodiment illustrated in FIGS. 9 and 10, at least a portion of outer surface 174 of core pin 190 near second end 178 may also include a second set of threads 182 that mate with corresponding threads 124 formed in inner bore 156 proximate distal portion 130 and after expandable portion 152. In this configuration, first and second set of threads 180, 182 of core pin 190 would mate into threads 122, 124, respectively, in inner bore 156 on both sides of expandable portion 152 to completely bridge expandable portion 152 to, for example, offload expandable portion 152 during insertion, as described in more detail below.

FIG. 4 illustrates bone fixation screw 100 in an unstressed or relaxed state and FIG. 5 illustrates bone fixation screw 100 in a stressed or unrelaxed state. Bone fixation screw 100 may be employed into a stressed or unrelaxed state at a predetermined force level by, for example, loading bone fixation screw 100 into a vice, stationary chuck or similar device that could stretch expandable portion 152 of intermediate portion 150 with a specific amount of force. This displacement could then be held or maintained by inserting core pin 170 of a predetermined length into inner bore 156 until second end 178 of core pin 170 touches far side 157 of the cannulation of inner bore 156 proximate distal portion 130. Core pin 170 maintains this displacement and prevents expandable portion 152 from returning to its pre-stretched or relaxed position. Since the displacement is held or maintained by core pin 170, the amount of force that caused the displacement is retained as pre-tension.

Alternatively, bone fixation screw 100 may be employed into a stressed or unrelaxed state by inserting core pin 170 using first set of threads 180 screwed into the threads 122 in inner surface of inner bore 156 proximate head portion 110. In this example, illustrated in FIG. 6, as core pin 170 is threaded down into inner bore 156 with second end 178 of core pin 170 abutting against far side 157 of the cannulation of inner bore 156 proximate distal portion 130, expandable portion 152 stretches as second end 178 pushes against far side 157 of the cannulation of inner bore 156 proximate distal portion 130.

As expandable portion 152 stretches, the amount of force required to continue to stretch goes up requiring an increasing amount of torque to continue to turn the core pin 170. One can calculate the torque level that produces a specific stretch force by using a torque to linear force equation inputting the pitch and diameter of the core pin threads. By using a torque limiting driver set to this predetermined torque value one can repeatedly set the tension force of the screw to the desired force.

There are various force levels that are desired based on known and available research. The desired force level may depend on, for example, the hardness and softness of the bone. Specific procedures may also call out for specific amounts of force based on known and available studies and research, so a bone constructed in accordance with one or more aspects of the present invention may be specifically design to a desired force level specific to the surgery. In one example, a bone fixation device 100 constructed in accordance with one or more aspect of the present may be stretched or expanded approximately two millimeters to create a pre-tension of forty pounds.

In one example, a bone fixation screw constructed in accordance with one or more aspects of the present invention may be pre-tensioned by a manufacturer to, for example, twenty pounds, thirty pounds, forty pounds, etc., and then packaged so that it would already be set for the amount of force needed by a surgeon. The screw could be stretched in a clean room to a desired force level and then a core pin 170 inserted to hold that force level.

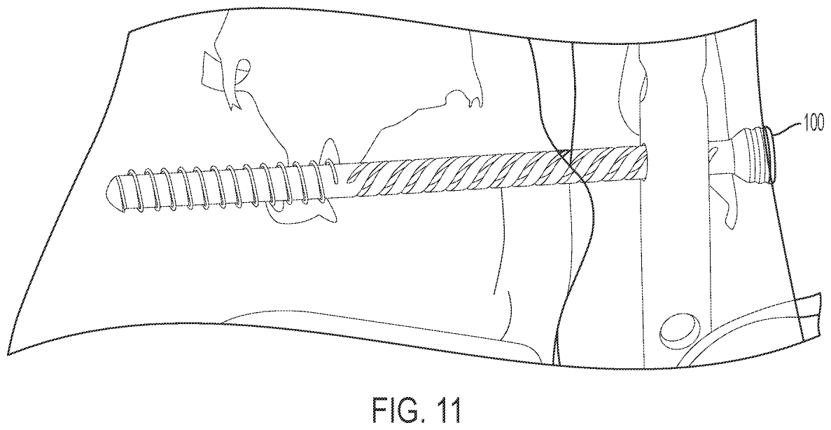

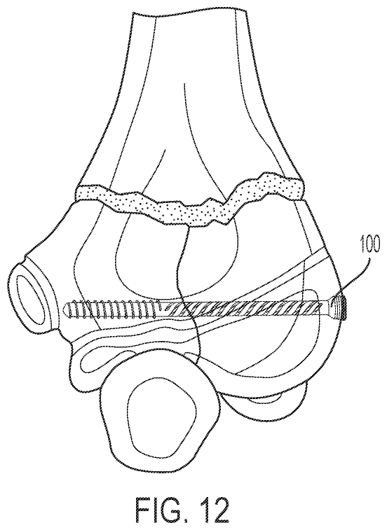

In one example of use of bone fixation screw 100 when it is desired to fasten or secure two or more bone fragments or members together (see, e.g., FIGS. 11 and 12), a pair of aligned bores may be predrilled or formed in the two bone fragments. Thereupon, bone fixation screw 100, in a predetermined stressed or unrelaxed position, containing core pin 170 is inserted into the first bore and a suitable tool, such as a hex driver, is inserted into recess 116 of head portion 110 to rotate distal portion 130 into the bone fragments. With continued insertion of bone fixation screw 100, distal portion 130 eventually enters and advances along the second bore of the distal bone fragment. Core pin 170 continues to provide stability to bone fixation screw 100 as advanced or inserted into the bone fragments. Continued rotation of the hex drive will not cause expandable portion to relace and contract because the core pin is holding it firmly in place. As distal portion 130 continues to advance along the distal bore of the second bone fragment, head portion 110 moves into engagement with, e.g. abuts, in one example, the outer surface of the proximal bone fragment or, alternatively, the top surface of a bone plate, such that continued advancement of distal portion 130 is prohibited.

At this point, bone fixation screw 100 contains the maximum amount of initial tension, and is comparable to the amount of initial tension achieved with a conventional bone screw. Once the bone fragments are fully apposed and bone fixation device 100 is fully inserted or seated, core pin 170 may be removed. When core pin 170 is removed, the pre-tension that was being held by core pin 170 is then transferred to the fragmented bones because the bones are now preventing bone fixation screw 100 from going back to its original length. In this configuration, the pre-tension force is effectively across the bone fragments.

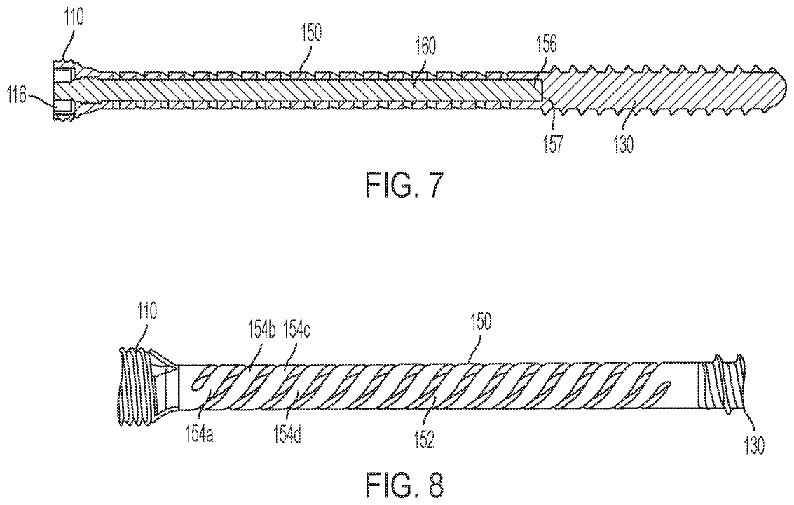

Once bone fixation screw 100 has been inserted and core pin 170 removed, bone fixation screw 100 has transitioned the pre-tension load to the fragmented bones. To avoid having core pin 170 carry any of the load that is now being held by the fragmented bones, a shorter core pin 192, as illustrated in FIG. 7, may be reinserted into inner bore 156 to alter or change the stiffness level of bone fixation screw 100. Shorter core pin 192 would be, in some examples, one to two millimeters shorter than core pin 179 that was in to hold it at the initial desired force level. Shorter core pin 192 would need to be short enough so as to not touch far side 157 of the cannulation of inner bore 156. If shorter core pin 192 is touching far side 157 of the cannulation of inner bore 156, shorter core pin 192 would be taking up some of the pre-tension force and not putting that force on the bones. When shorter core pin 192 is not touching far side 157 of the cannulation of inner bore 156, then expandable portion 152 is being held to length by the bones only, and, therefore, being subjected to all of the pre-tension force.

Depending on the stiffness of shorter core pin 192, the overall stiffness of bone fixation screw will change. For example, if a more flexible bone fixation screw is desired, core pin(s) may be left out all together or a shorter core pin made from a more flexible material, such as, for example, polyethylene or PEEK, may be employed. Alternatively, if a stiffer bone fixation screw is desired, core pin(s) may be made from different metals, including, for example, a soft titanium allow to a very hard CoCrMo alloy.

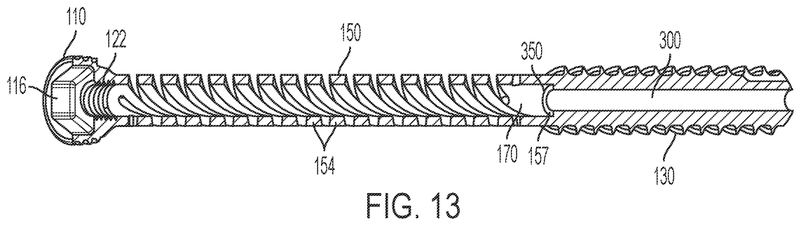

In an alternative embodiment as illustrated in FIG. 13, a bone fixation screw constructed in accordance with one or more aspects of the present invention may include a cannulation 300 extending the length of bone fixation screw 100 from recess 116 to the end of distal portion 130 to allow the screw to be inserted over, for example, k-wire. In this example, far side 157 of inner bore 156 is formed by a step transition 350 from inner bore 156 to the rest of cannulation 300 extending into distal portion 130.

A bone fixation screw constructed in accordance with one or more aspects of the present invention achieves several advantages over conventional and existing bone fixation screws or devices. For example, a bone fixation screw including an expandable portion and removeable core pins constructed in accordance with one or more aspects of the present invention provides a finite amount of compression that is known to be optimal clinically.

While several aspects of the present invention have been described and depicted herein, alternative aspects may be affected by those skilled in the art to accomplish the same objectives. Accordingly, it is intended by the appended claims to cover all such alternative aspects as fall within the true spirit and scope of the invention.

What is claimed:

1. A bone fixation screw, said bone fixation screw comprising:
a head portion, said head portion including an end surface, the end surface forming an opening;
a distal portion;
an intermediate portion, said intermediate portion extending between said head portion and said distal portion, said intermediate portion including tubular body, the tubular body including an expandable portion and defining an inner bore, the inner bore in communication with the opening formed in said head portion;
a core pin, said core pin removeable coupled within the inner bore of said intermediate portion.

2. The bone fixation screw of claim 1, wherein said core pin includes a first end and a second end, and an outer surface, the outer surface proximate the first end including a first set of threads for mating with a second set of threads on the inner bore proximate said head portion.

3. The bone fixation screw of claim 2, wherein the inner bore terminates at an end beyond the expandable portion of said intermediate portion proximate said distal portion.

4. The bone fixation screw of claim 3, wherein the second end of said core pin abuts against the end of the inner bore to expand the expandable portion as core pin is advanced within the inner bore by the mating first and second set threads on the outer surface of said core pin and the inner bore proximate said head portion.

5. The bone fixation screw of claim 2, wherein the outer surface proximate the second end of said core including a third set of threads for mating with a fourth set of threads on the inner bore proximate said distal portion.

6. The bone fixation screw of claim 5, wherein the expandable portion of said intermediate portion is between the second set of threads and the fourth set of threads on the inner bore of said intermediate portion.

7. The bone fixation screw of claim 6, wherein the expandable portion stretches between a rested position and an unrested position.

8. The bone fixation screw of claim 7, wherein said core pin is removably coupled within the inner bore when the expandable portion is in the unrested position.

9. The bone fixation screw of claim 8, further comprising a second core pin, said second core pin removably coupled within the inner bore of said intermediate portion in place of said core pin, said second core pin being shorter in length than said core pin.

10. A method of securing a bone member, said method comprising:
forming at least one bore in the bone member;
providing a bone fixation screw, said bone fixation screw including:
a head portion, said head portion including an end surface, the end surface forming an opening;
a distal portion;
an intermediate portion, said intermediate portion extending between said head portion and said distal portion, said intermediate portion including tubular body, the tubular body including an expandable portion and defining an inner bore, the inner bore in communication with the opening formed in said head portion; and
a first core pin;
expanding said bone fixation device to an unrest position with a predetermined amount of force;
inserting and removeably coupling said core pin within the inner bore of said intermediate portion;
inserting said bone fixation screw into the at least one bore; and
removing said core pin from the inner bore of said intermediate portion.

11. The method of claim 10, further comprising inserting and removeably coupling a second core pin within the inner bore of said intermediate portion, wherein said second core pin is shorter in length than said first core pin.
